(19) United States
(12) Patent Application Publication
(10) Pub. No.: US 2005/0251227 A1
(43) Pub. Date: Nov. 10, 2005

(54) Transferring software over a wireless radio frequency link onto external programmers for implantable medical devices

(75) Inventors: Heng/Jim Khoo, Saint Paul, MN (US); Gang Wu, Shoreview, MN (US)

Correspondence Address: Merchant & Gould P.C., P.O. Box 2903, Minneapolis, MN, 55402-0903, US

(73) Assignee: Cardiac Pacemakers, Inc.

(21) Appl. No.: 10/838,715

(22) Filed: May 4, 2004

(51) Int. Cl. A61N 1/08

(52) U.S. Cl. 607/60

(57) ABSTRACT

Methods, computer program products, and systems are provided for transferring software from a computing device over a high-speed wireless radio frequency (RF) link onto one or more external programmers of implantable medical devices. The methods include entering a communications range of the external programmers and establishing communication over the wireless RF link. The method may also include determining whether any external programmers are without the software stored on the computing device. In response to determining an external programmer is without the software, the computing device uploads and the external programmer receives and stores the software over the wireless radio frequency link without interrupting the functionality of the external programmer while the programmer interfaces with an implantable medical device.

### TECHNICAL FIELD

The present invention is related to software transfer features of a computing system. More particularly, the present invention is related to uploading software onto one or more external programmers for implantable medical devices via a high-speed wireless radio frequency (RF) link.

### BACKGROUND OF THE INVENTION

Reducing the time, risk, and labor of software upload or transfer operations on computing devices, such as external programmers for implantable medical devices, is an ongoing objective of implant medical device manufacturers. Previous methods for transferring software onto external programmers require direct physical contact or human intervention with external programmers by inserting a physical media, such as a disk, one at a time into each external programmer that requires an update. Thus, these previous methods necessitate a data storage or data transfer peripheral on each external programmer, sending out an update physical media to field personnel, and/or potential reliance on a third party physical media that may be prone to obsolescence depending on the physical media's success in the consumer market.

In one previous method, a company's corporate center may send out a physical media to field personnel in the form of compact disks (CDs) or other formats. After receiving the CD, field personnel turn on each external programmer, insert the CD into a data storage or data transfer peripheral, and launch a program to transfer or copy the software from the CD onto the external programmer. Removing the need to physically contact or manually intervene with each external programmer one at a time in preparation to transfer software is especially useful for a pool or group of external programmers that need the same software update and/or trial. For instance, manually updating a large group of external programmers one at a time can take an excessive amount of time and administrative labor. Although previous methods of wireless software transfer are capable of downloading firmware to a FLASH EEPROM of an external device, these methods utilize inductive telemetry. Inductive telemetry still necessitates one at a time manual intervention and functionality interruptions with each external device as well as a relative close proximity between the transferring device and the external device and a relatively slow transfer speed.

It is with respect to these considerations and others that the present invention has been made.

### SUMMARY OF THE INVENTION

In accordance with embodiments of the present invention, the above and other problems are solved by methods, systems, and computer program products for transferring software onto one or more computing devices, such as external programmers for implantable medical devices, over a wireless radio frequency (RF) link. For the purposes of describing the present invention, software includes computing device instructions, data, and anything else that can be stored electronically. Embodiments of the present invention particularly relate to transferring software from a portable computing device, for example a Personal Digital Assistant (PDA) or a laptop computer, onto individual or a pool of external programmers over a wireless RF link. The portable computing device may acquire the software to be transferred from another portable computing device and/or a secured corporate server via a network connection. The portable computing device may then be brought within wireless RF range of a pool of external programmers, determine which programmers are without the software to be transferred, and transfer the acquired software via a wireless RF link onto one or more of the external programmers. The software transfer may occur without interrupting any functionality while the external programmers are in use, such as when interfacing with implantable medical devices.

One embodiment provides a method for transferring software from a transfer computing device, such as a PDA, onto one or more computing devices, such as external programmers, over a wireless RF link. The method involves establishing communication with the computing devices over the wireless RF link. Communication may be established by entering a wireless RF communications range of the computing devices and executing a software transfer application on the transfer computing device. Once communication is established, the software is uploaded from the transfer computing device onto at least one of the computing devices over the wireless RF link. Computing devices designated to receive the software transfer may be identified by determining, over the wireless RF link, whether any of the computing devices are without the software. Thus, the need for manual insertion of a portable media into each computing device in order to transfer the software is eliminated.

Another embodiment is a method for transferring software from a computing device via a wireless RF link onto one or more external programmers for implantable medical devices. The method involves receiving communication at the external programmers from the portable computing device over the wireless RF link. The external programmers then prepare to receive an upload of the software via the wireless RF link. Next, the upload of the software is received from the computing device via the wireless RF link. Finally, the software is stored onto a memory of the one or more external programmers for future use.

Still another embodiment is a computer program product comprising a computer usable medium having control logic stored therein for causing a computer to transfer software onto one or more external programmers for implantable medical devices over a wireless radio frequency (RF) link. The control logic includes computer readable program code for causing the computer to establish communication with the external programmers over the wireless RF link, determine whether any of the external programmers are without the software; and upload the software from the computer onto at least one of the external programmers over the wireless radio frequency link in response to determining that the at least one of the external programmers is without the software. Software may include external programmer instructions and/or data.

Another embodiment is a computer program product comprising a computer usable medium having control logic stored therein for causing external programmers for implantable medical devices to receive software via a wireless RF link from a computing device. The control logic includes computer readable program code for causing the external programmers to receive communication from the computing device via the wireless RF link and prepare to receive an upload of the software via the wireless RF link. The control logic further includes computer readable program code for causing the external programmers to receive the upload of the software from the computing device via the wireless RF link and store the software onto a memory of each external programmer receiving the upload of software.

Another embodiment of the present invention is a system for transferring software from a computing device onto one or more external programmers for implantable medical devices over a wireless radio frequency (RF) link. The system includes at least one external programmer operative to receive communication from the computing device via the wireless RF link and in response to receiving the communication, detect whether any version of the software and/or what version of the software is currently stored on the external programmer receiving the communication. The external programmer is also operative to communicate to the computing device via the wireless RF link whether any version of the software and/or what version of the software is currently stored on each external programmer receiving the communication. Still further, the external programmer is operative to receive the upload of the software from the computing device via the wireless RF link and store the software onto a memory of each external programmer receiving the upload of software.

The system also includes the computing device operative to establish communication with the external programmer via the wireless RF link, determine whether the external programmer is without the software, and upload the software onto the external programmer via the wireless radio frequency link in response to determining that the external programmer is without the software.

Aspects of the invention may be implemented as a computer process, a computing system, or as an article of manufacture such as a computer program product or computer-readable medium. The computer program product may be a computer storage media readable by a computer system and encoding a computer program of instructions for executing a computer process. The computer program product may also be a propagated signal on a carrier readable by a computing system and encoding a computer program of instructions for executing a computer process.

These and various other features as well as advantages, which characterize the present invention, will be apparent from a reading of the following detailed description and a review of the associated drawings.

### BRIEF DESCRIPTION OF THE DRAWINGS

FIG. 1 illustrates a networked and wireless RF operating environment for embodiments of the present invention that allow a portable computing device to transfer software over a wireless RF link to one or more external programmers for implantable medical devices;

FIG. 2 is a block diagram illustrating the operating environment of FIG. 1 and system architecture components utilized in embodiments of the invention;

FIG. 3 illustrates a computing system architecture for an external programmer utilized in embodiments of the invention;

FIG. 4 illustrates a computing system architecture for a computing device utilized in embodiments of the invention; and

FIG. 5 illustrates an operational flow performed in transferring software from a computing device onto one or more external programmers via a wireless RF link according to an embodiment of the invention.

### DETAILED DESCRIPTION

As described briefly above, embodiments of the present invention provide methods, computer program products, and systems for transferring software onto one or more computing devices, such as external programmers for implantable medical devices, via a wireless RF link. In the following detailed description, references are made to accompanying drawings that form a part hereof, and in which are shown by way of illustration specific embodiments or examples. These embodiments may be combined, other embodiments may be utilized, and structural changes may be made without departing from the spirit and scope of the present invention. The following detailed description is, therefore, not to be taken in a limiting sense, and the scope of the present invention is defined by the appended claims and their equivalents.

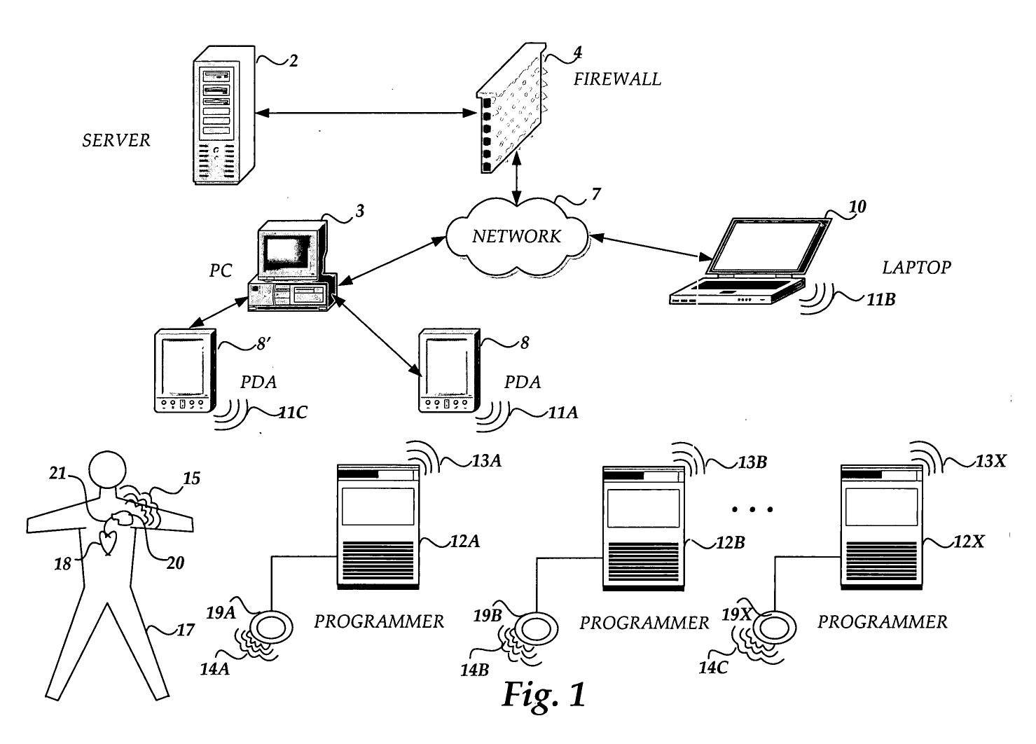

Referring now to the drawings, in which like numerals represent like elements through the several figures, aspects of the present invention and the exemplary operating environment will be described. FIGS. 1-4 and the following discussion re intended to provide a brief, general description of a suitable computing environment in which the embodiments of the invention may be implemented. While the invention will be described in the general context of program modules that execute to transfer software, via a wireless RF link, onto external programmers for implantable medical devices from a portable computing device, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

Referring now to FIG. 1, a networked and wireless RF operating environment for embodiments of the present invention that allow a portable computing device to transfer software over a wireless RF link to one or more external programmers for implantable medical devices will be described. As shown in FIG. 1, the operating environment includes portable transfer computing devices, a PDA 8 and/or a laptop computer 10, which may receive the software to be transferred from a secure server computer 2 via the network 7. For instance, the PDA 8 may dock with a personal computer (PC) 3 connected to the network 7, such as the Internet or Intranet. The PC 3 may download the software to be transferred from the secure server 2, secured by a firewall 4. Alternatively, the laptop computer 10 may be connected to the network 7 and directly download the software to be transferred from the secure server computer 2. Also alternatively, instead of the PDA 8 receiving the software to be transferred by being docked to the PC 3, the PDA 8 may receive the software from a second PDA 8′ storing the software to be transferred. This exchange may occur through a wireless RF signal 11C received at the PDA 8 from the PDA 8′ or through other peer-to-peer communication means.

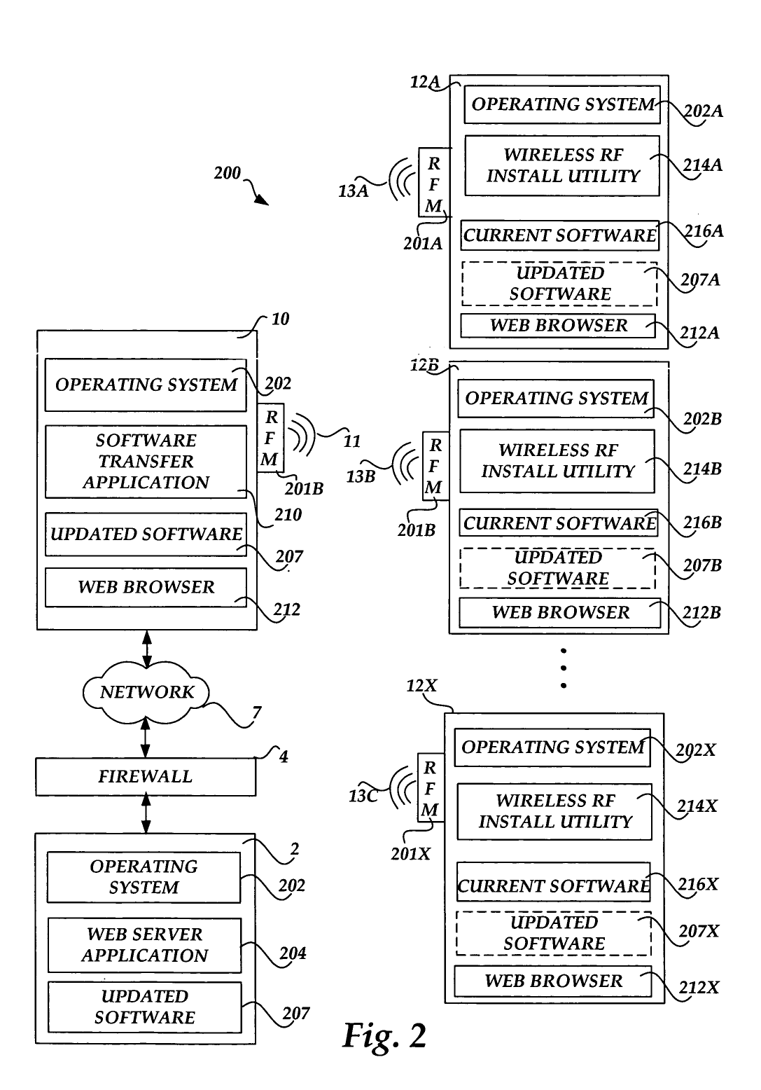

Once the software is stored on the computing device 8 and/or 10, the PDA 8 and/or the laptop 10 may establish wireless RF communication with and transfer the software to one or more external programmers 12A-12X by radiating a wireless RF signal 11A or 11B and receiving the wireless RF signals 13A-13X respectfully produced by the external programmers 12A-12X. The computing devices 8 and/or 10 may also interrogate the external programmers 12A-12X via the wireless RF signals 11A-11B to determine which external programmers are without the software to be transferred. Additional details regarding components involved in transferring software will be described below with respect to FIGS. 2-4.

Subsequently, once the software is transferred to one or more of the external programmers, the software may be stored and utilized while any of the external programmers interface with an implantable medical device 20, such as an implantable pulse generator. The medical device 20 is implanted in a patient 17 and coupled to the heart 18 by one or more leads 21. The external programmers 12A-12X are adapted to be communicatively coupled to the medical device 20 to receive and transmit control commands, program instructions, and cardiac data to and from the medical device 20 via loop antennas 19A-19X that respectively radiate inductive signals 14A-14X and receives an inductive signal 15 produced by the medical device 20. The external programmers 12A-12X are used in application to various activities such as electrical lead 21 placement and implantable device 20 optimization.

Turning now to FIG. 2, a block diagram illustrating the operating environment of FIG. 1 and system architecture components utilized in embodiments of the invention will be described. As shown in FIGS. 1 and 2, the system 200 includes a computing device, such as the laptop computer 10 or the PDAs 8 and/or 8′. For the purposes of describing the invention, the laptop 10 will be used in the description below. The laptop computer 10 comprises a standard portable computer that is operative to execute a software transfer application 210 and includes wireless capability. Wireless RF capability may be in the form of an RF module transceiver (RFM) 201. Alternatively, wireless capability may be built into the laptop computer 10 or may be part of a removable PCMCIA or COMPACTFLASH CARD from SANDISK CORPORATION.

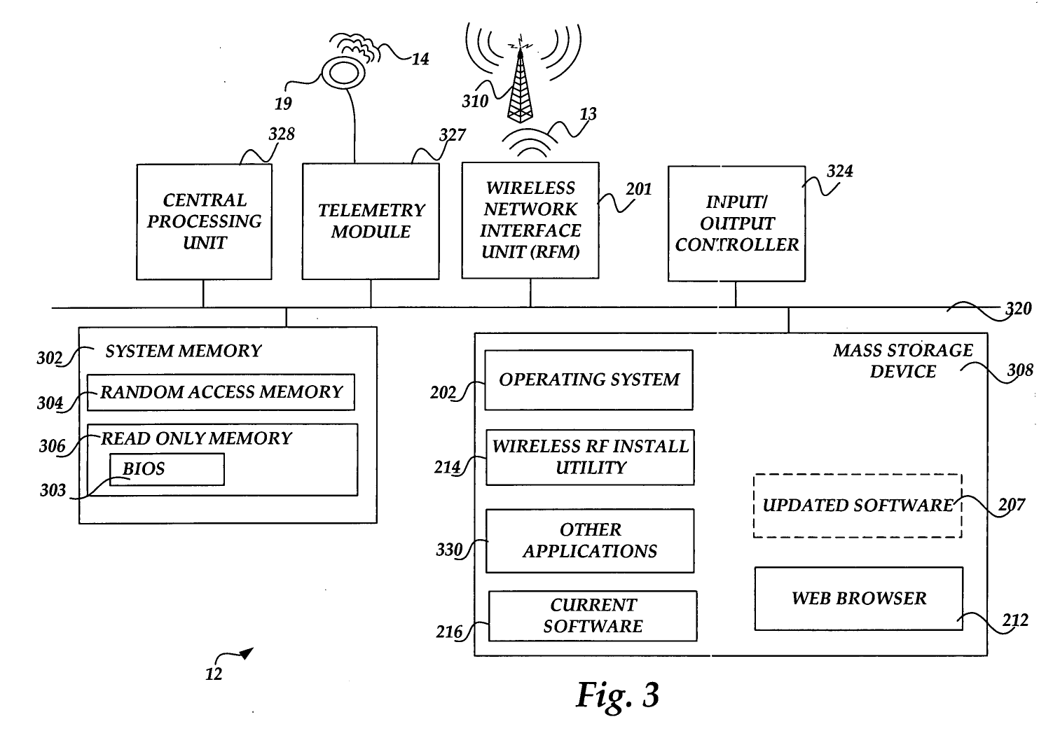

The software transfer application 210 may operate in conjunction with an operating system 202, a web browser 212, and the RFM 201 to transfer or copy the updated and/or trial software 207 to the external programmers 12A-12X via the wireless RF link. As will be described in greater detail below with respect to FIGS. 3-5, the wireless RF signals 13A-13X contain data that assists the software transfer application 210 in identifying which external programmers 12A-12X are without the updated software 207.

The laptop computer 10 may be connected to the network 7, such as a LAN, WAN, or other type of distributed computing network, such as the Internet, that supports the transmission control protocol/Internet protocol (“TCP/IP”). It should be appreciated, however, that the laptop computer 10 may be configured for communication over other types of networks. Alternatively, the laptop computer 10 may comprise another type of computing device operative to access the network 7, such as a handheld computing device, a PDA, and a pocket PC.

As briefly described above with respect to FIG. 1, the laptop computer 10 may download the updated software 207 from the secure server computer 2 to be transferred to the external programmers 12A-12X via the wireless RF signal 11 for use in updating current software 216A-216X presently stored respectively on the external programmers 12A-12X. The updated software 207 may be a trial or an updated version of the current software 216A-216X. Transfer of the updated software 207 onto the external programmers 12A-12X may be performed in order to update the current software 216A-216X after the version has been changed, corrupted and/or erased. In particular, the updated software 207 is remotely transferred from the laptop computer 10 via the wireless RF signal 11 to the memory of the external programmers 12A-12X that provide the signal 13A-13X indicating a need for the updated software 207.

The external programmers 12A-12X may comprise a pool of implantable medical device programmers in use or on standby in a hospital. The external programmers 12A-12X are also respectively equipped with RFMs 201A-201X and are operative to execute wireless RF install utilities 214A-214X, respectively. The wireless RF install utilities 214A-214X operate respectively in conjunction with operating systems 202A-202X to monitor and/or respond to wireless RF communication such as from the wireless RF signal 11. The software transfer application 210 may send a software version inquiry via the wireless RF signal 11 to the wireless RF install utilities 214A-214X respectively executing on the external programmers 12A-12X. In the alternative, the wireless RF install utilities 214A-214X may not be executing but may be prompted upon the operating systems 202A-202X detecting wireless RF communication from the wireless RF signal 11.

Once a wireless RF install utility, such as 214A, receives a version inquiry from the software transfer application 210, the wireless RF install utility 214A detects whether the current software 216A is of a different version than the updated software 207 to be transferred or whether any version of the updated software is stored. In the alternative, the wireless RF install utility may detect and communicate, via the wireless RF signal 13A, the version presently stored as the current software 214A. The software application 210 will then determine whether the stored current software 216A is different from the updated software 207.

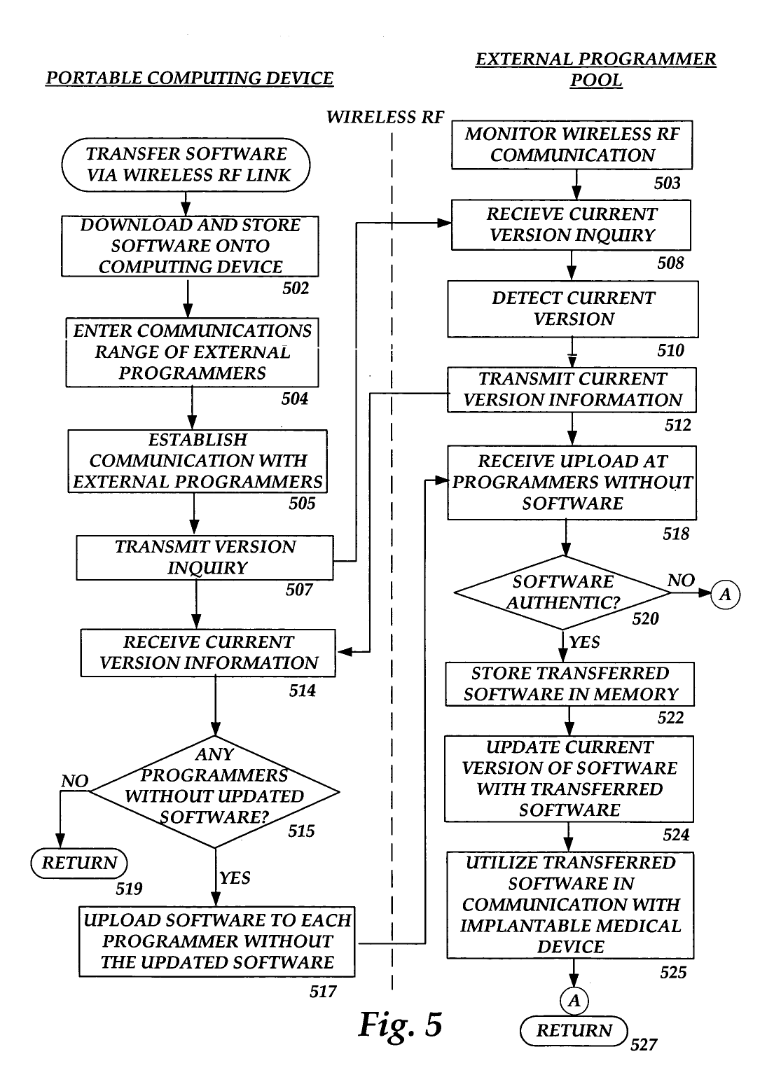

Upon determining that any of the external programmers 12A-12X is without the updated software 207, the software transfer application 210, via the wireless RF signal 11, transfers the updated software 207 onto the external programmers 12A-12X presently without the updated software 207. The signal 11 propagates over the wireless RF link to the corresponding RFM 201A-201X for installation within the memory of the external programmers without the updated software 207. The updated software 207A-207X are illustrated in hashed-blocks to show that when any of the external programmers 12A-12X have a current software that matches the updated software version, the updated software 207 will not be transferred and stored on those external programmers. Additional details regarding transmission of the updated software 207 will be described below with respect to FIG. 5.

FIG. 3 shows a block diagram of an external programmer 12 computing device incorporating input and output communication functions. The external programmer 12 includes a communications device such as a telemetry module 327, a central processor 328, a system memory 302, and a system bus 320 that couples the system memory 302 to the central processor 328. The system memory 302 includes read-only memory (ROM) 306 and random access memory (RAM) 304. A basic input/output system 303 (BIOS), containing the basic routines that help to transfer information between elements within the external programmer 12, such as during start-up, is stored in ROM 306. The external programmer 12 further includes a mass storage device (MSD) 308 for storing an operating system 202 such as WINDOWS XP, from MICROSOFT CORPORATION of Redmond, Wash., other applications 330, such as a programming interface application, and a web browser application 212 for example INTERNET EXPLORER from MICROSOFT CORPORATION of Redmond, Wash. It should be appreciated that a web browser is not necessary in lieu of other software, such as proprietary software capable of supporting a TCP/IP or other protocol stack. The MSD 308 may also store the wireless RF install utility 214 which in cooperation with the processor 328 is operative to receive and transmit wireless RF communication, detect the version of the current software 216, and install the updated software 207 when transferred.

The MSD 308 is connected to the central processor 328 through a mass storage controller (not shown) connected to the system bus 320. The MSD 308 and its associated computer-readable media, provide non-volatile storage for the external programmer 12. Although the description of computer-readable media contained herein refers to a mass storage device, such as a hard disk or CD-ROM drive, it should be appreciated by those skilled in the art that computer-readable media can be any available media that can be accessed by the CPU 328.

The telemetry module 327 receives signals from and sends signals to the central processor 328 through the signal bus 320. Telemetry module 327 also sends to and receives signals from the loop antenna 19, which typically is a wire loop. The telemetry communications device 327 may use circuitry such as that known in the art for implantable device communications. The input/output controller 324 may also be included with the programmer 12 for receiving and processing input from a number of input devices (not shown). The input/output controller 324 communicates with the processor 328 through the system bus 320. It should be appreciated that the external programmer 12 may be without local floppy storage or serial port access which is a necessity for previous systems to transfer software.

The laptop computer 10 radiates the wireless RF signal 11 that has encoded information, such as the updated software 207 being transferred onto the external programmer 12. The radiated signal 11 propagated from the laptop computer 10 is received by a network interface unit, such as the RFM 201 and is converted to an electrical signal that is transferred to the telemetry module 327. The telemetry module 327 may then employ an analog-to-digital conversion to convert the received signal to a data signal that is then passed to the central processor 328.

The central processor 328 may employ various operations, discussed in more detail below with reference to FIG. 5 to provide and utilize the signals propagated between the external programmer 12 and the telemetry module 327. The processor 328 may store data to and access data from mass storage device 308, such as electronic memory or magnetic storage. Data is transferred to and received from the storage device 308 through the system bus 320. The processor 328 may be a general-purpose computer processor or processor typically used for an external programmer. Furthermore as mentioned below, the processor 328, in addition to being a general-purpose programmable processor, may be firmware, hard-wired logic, analog circuitry, other special purpose circuitry, or any combination thereof.

According to various embodiments of the invention, the programmer 12 operates in a networked and wireless RF environment, as shown in FIGS. 1 and 2, using logical connections to remote computing devices via wireless RF communication, such as an Intranet, or a local area network (LAN). The programmer 12 may connect to a wireless RF link 310 via a wireless network interface unit, such as the RF module transceiver 201 connected to the system bus 320 and radiating the wireless RF signal 13. It should be appreciated that the wireless network interface unit 201 may also be utilized to connect to other types of networks and remote computer systems.

A computing device, such as the external programmer 12, typically includes at least some form of computer-readable media. Computer readable media can be any available media that can be accessed by the computing system 12. By way of example, and not limitation, computer-readable media might comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, RAM, ROM, EPROM, flash memory or other memory technology, CD-ROM, digital versatile disks (DVD) or other optical storage, magnetic cassettes, magnetic tape, magnetic disk storage or other magnetic storage devices, or any other medium that can be used to store the desired information and that can be accessed by the computing system 12.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media. Computer-readable media may also be referred to as computer program product.

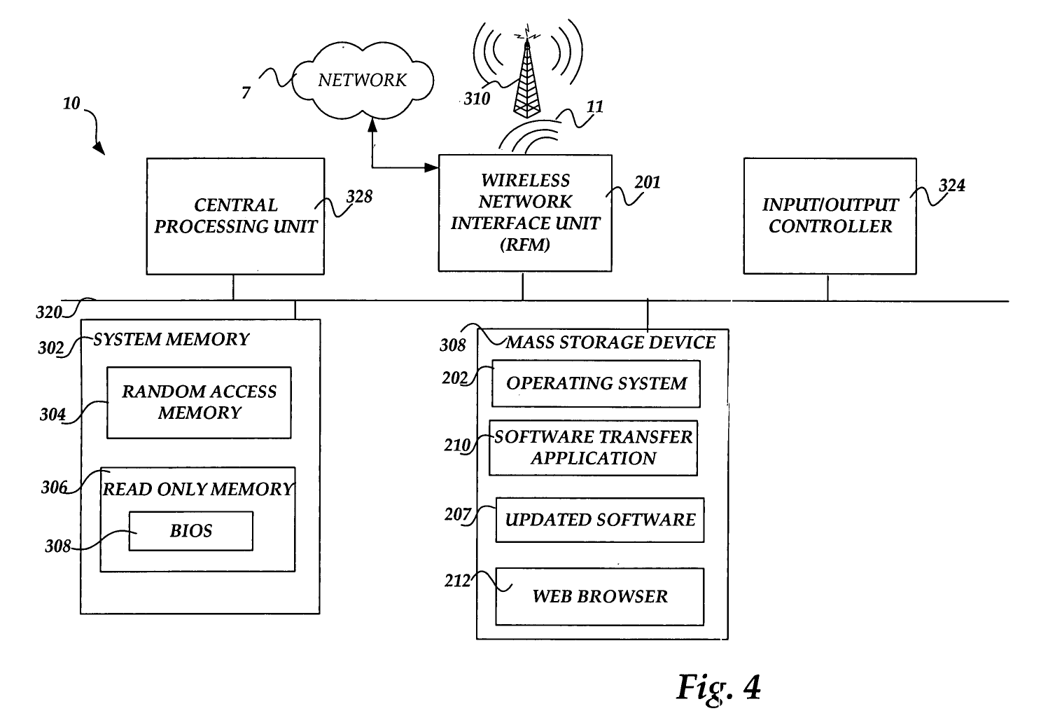

Turning now to FIG. 4, a computing system architecture for a computing device, such as the laptop computer 10 or PDAs 8 and/or 8′ utilized in embodiments of the invention will be described. The computer architecture shown in FIG. 4 illustrates a conventional portable computing device, including a CPU 328 and a system memory 302 containing components described above with respect to FIG. 3. The mass storage device 308 may also store the updated software 207 and the web browser 212. The laptop computer 10 also includes a mass storage device 308 for storing the operating system 202 and application programs, such as the software transfer application 210 for initiating and transferring the stored updated software 207 to the external programmers 12A-12X via the wireless RF link 310 and the wireless RF signal 11. The laptop computer 10 may also be operative to execute a Web browser application 212 stored on the mass storage device 308 as described above with respect to FIGS. 2-3.

Turning now to FIGS. 1, 2, and 5, an illustrative operational flow 500 for transferring software via a wireless RF link onto at least one of the external programmers 12A-12X according to an embodiment of the present invention will be described. It should be appreciated that the number of external programmers 12A-12X having a wireless RF link may vary and may be in use or on standby without being interrupted by the transfer operations. The operational flow begins at operation 502 where the computing device, such as the laptop computer 10 and/or the PDAs 8 and/or 8′, downloads and stores the updated software 207 to be transferred from the secure server computer 2 over the network 7 onto the computing device/laptop 10.

The operational flow 500 then continues to operation 504 where the laptop computer 10 enters a communications range of any of the external programmers 12A-12X. It should be appreciated that the communication range and speed of wireless RF telemetry signals are by far greater than that of inductive telemetry signals, such as the inductive telemetry signal 14A described above. Thus, wireless RF communication is faster and propagates over greater distances than inductive telemetry communication.

The operational flow 500 then continues to operation 505 where the software transfer application 210 establishes wireless RF communication with at least one of the external programmers by radiating the wireless RF signal 11. Next, the software transfer application determines which external programmers 12A-12X are without the updated software 207 by transmitting a version inquiry at operation 507. The RFM 201, prompted by the software transfer application 210, transmits the version inquiry via the wireless RF signal 11. Meanwhile, at operation 503, the external programmers 12A-12X monitor the wireless RF link via the RFMs 201A-201X working in conjunction with the operating systems 202A-202X and/or the wireless RF install utilities 214A-214X, and the web browsers 212A-212X. It should be appreciated that the web browser may be substituted with proprietary software for wireless RF communications.

Next, at operation 508, the wireless RF signal is received at at least one of the RFMs 201A-201X, such as the RFM 201A. The operational flow then continues to operation 510 where the wireless RF install utility 214A detects whether any or what current version 216A of the software is presently stored. The operational flow 500 then continues to operation 512 where the wireless RF install utility transmits current version information to the laptop computer 10 via the wireless RF signal 13A. It should be appreciated that the wireless RF signal 13A identifies what external programmer is transmitting the current version information. This identification assists the software transfer application in determining which external programmers are without the updated software 207.

The operational flow then continues to operation 514 where the software transfer application 210 receives the current version information. Then at operation 515, either upon receiving the current information from all the external programmers 12A-12X or one at a time, the software transfer application 210 determines which external programmers 12A-12X are without the updated software 207. If all the external programmers transmit as having the updated software 207 already, the operational flow 500 continues from operation 515 to return operation 519 where control is passed to other routines. However, if any external programmers are without the updated software 207, operational flow 500 continues from operation 515 to operation 517. At operation 517, the software transfer application 210 uploads the updated software 207 to each external programmer 12A-12X without the updated software 207 via the wireless RF signal 11.

Next, at operation 518, the wireless RF install utilities 214A-214X for each external programmer 12A-12X not storing the updated software 207 receives the upload of software. Then operational flow 500 continues to operation 520 where the authenticity of the updated software 207 uploaded is verified. If the updated software 207 is not authentic, operational flow 500 terminates at return operation 527. If the updated software 207 is authentic, the operational flow 500 continues from operation 520 to operation 522. At operation 522, the updated software 207 transferred is stored in a memory of the external programmer receiving the upload.

Next, at operation 524, the current software 216A-216X is replaced or updated with the updated software 207 for each external programmer 12A-12X receiving the upload. This may occur respectively upon the reset and/or reboot of an external programmer. Finally, at operation 525, the updated software 207 transferred, may be utilized while the external programmer is in use interfacing with the implantable medical device 20. The operational flow then terminates at return operation 527 where control is passed to other routines.

It should be appreciated that security measures known in the art may be used to prevent unsecured access to transfer and update operations. For example, downloads may be executed behind a secure firewall and in encrypted communication formats and authentication credentials may be implemented and utilized to control security.

Thus, the present invention is presently embodied as methods, systems, computer program products or computer readable mediums encoding computer programs for transferring software onto one or more computing devices, such as external programmers for implantable medical devices, via a wireless RF link.

As various changes may be made in the above system elements, software modules and methods without departing from the scope of the invention, it is intended that all matter contained in the above description as shown in the accompanying drawings shall be interpreted as illustrative and not in a limiting sense. Since many embodiments of the invention can be made without departing from the spirit and scope of the invention, the invention resides in the claims hereinafter appended.

1. A method for transferring software from a transfer computing device onto one or more computing devices over a wireless radio frequency (RF) link comprising:
establishing communication with the computing devices over the wireless RF link; and
uploading the software from the transfer computing device onto at least one of the computing devices over the wireless radio frequency link thereby eliminating the need for manual insertion of a portable media into each computing device in order to transfer the software.

2. The method of claim 1, further comprising determining whether any of the computing devices are without the software, wherein uploading the software occurs in response to determining that the at least one of the computing devices is without the software wherein the software comprises at least one of computing device instructions and data.

3. The method of claim 1, further comprising:
accessing a network to retrieve the software from a secured remote server onto the transfer computing device; and
downloading the software onto the transfer computing device from the secured remote server over the network wherein the network is at least one of wired and wireless.

4. The method of claim 1, further comprising:
accessing a second transfer computing device storing the software to retrieve the software from the second transfer computing device onto the transfer computing device; and
downloading the software onto the transfer computing device from the second transfer computing device.

5. The method of claim 4, wherein the second transfer computing device comprises at least one of the following:
a handheld computing device;
a personal digital assistant (PDA);
a laptop computer; and
a pocket personal computer.

6. The method of claim 1, wherein the transfer computing device is portable and comprises at least one of the following:
a handheld computing device;
a personal digital assistant (PDA);
a laptop computer; and
a pocket personal computer.

7. The method of claim 1, wherein the computing devices comprise one or more external programmers for implantable medical devices.

8. The method of claim 7, wherein the external programmers are at least part of a pool of external programmers.

9. The method of claim 8, wherein uploading the software from the transfer computing device onto the at least one of the computing devices comprises transmitting the software to the at least part of the pool of external programmers in succession without manual intervention.

10. The method of claim 7, wherein uploading the software from the transfer computing device onto the at least one of the computing devices comprises transmitting the software to at least one external programmer without interrupting a functionality of the at least one external programmer while the at least one external programmer interfaces with at least one of the implantable medical devices.

11. The method of claim 2, wherein determining whether any of the computing devices are without the software comprises verifying whether each of the computing devices is storing a version of the software different from the software to be uploaded.

12. The method of claim 11, wherein each computing device is verified in succession.

13. The method of claim 11, wherein verifying whether each of the computing devices store a version of the software different from the software to be uploaded comprises:
transmitting an inquiry to each of the computing devices requesting whether any version of the software is presently stored on the computing device inquired;
receiving a response from each computing device as to what version is presently stored on the computing device; and
determining from each response, which of the computing devices is presently one of not storing any version of the software and storing a version of the software different from the software to be uploaded.

14. The method of claim 1, wherein uploading the software comprises transmitting a version of the software different from a version of the software presently stored on the at least one of the computing devices wherein the software is transmitted for storage on each computing device having one of no version of the software stored and a version of the software different from the software to be uploaded.

15. The method of claim 1, wherein the software is uploaded in an encrypted format for authentication at the at least one of the computing devices.

16. The method of claim 1, wherein communication between the transfer computing device and the computing devices is conducted in an encrypted format.

17. The method of claim 1, wherein establishing communication with the computing devices over the wireless RF link comprises entering a wireless RF communications range of the computing devices and executing a software transfer application on the transfer computing device wherein the software transfer application initiates transmission of communication signals over a high speed wireless RF link.

18. A method for transferring software from a computing device via a wireless RF link onto one or more external programmers for implantable medical devices comprising:
receiving at the one or more external programmers, communication from the computing device over the wireless RF link;
preparing to receive an upload of the software via the wireless RF link;
receiving the upload of the software from the computing device via the wireless RF link; and
storing the software onto a memory of the one or more external programmers.

19. Then method of claim 18, further comprising:
replacing the version of the software currently stored with the upload of the software; and
utilizing the software while in communication with the implantable medical devices.

20. The method of claim 18, wherein the communication received from the computing device comprises an inquiry as to which external programmers are without the software to be transferred from the computing device.

21. The method of claim 18, wherein preparing to receive the upload of software comprises:
in response to receiving the communication, detecting at least one of whether any version of the software and what version of the software is currently stored on the one or more external programmers; and
communicating to the computing device via the wireless RF link at least one of whether any version and what version of the software is currently stored on the external programmer.

22. The method of claim 20, wherein the one or more external programmers comprise a pool of external programmers and wherein the upload of the software is received at each external programmer within the pool without the software transferred from the computing device, in response to communicating which external programmers are without the software to be transferred from the computing device.

23. The method of claim 18, wherein the computing device is portable and comprises at least one of the following:
a handheld computing device;
a personal digital assistant (PDA);
a laptop computer; and
a pocket personal computer.

24. The method of claim 18, wherein the upload of the software is received in an encrypted format, further comprising:
determining whether the upload of software is authentic; and
in response to determining that the upload of software is authentic, storing the software onto a memory of the one or more external programmers.

25. The method of claim 18, further comprising at least one of monitoring the wireless RF communications link and receiving notice of a wireless RF communication and in response to receiving the notice, establishing communication with the computing device by executing a wireless communication application on the external programmers receiving the notice.

26. A computer program product comprising a computer usable medium having control logic stored therein for causing a computer to transfer software onto one or more external programmers for implantable medical devices over a wireless radio frequency (RF) link, the control logic comprising computer readable program code for causing the computer to:
establish communication with the external programmers over the wireless RF link;
determine whether any of the external programmers are without the software wherein the software comprises at least one of external programmer instructions and data; and
upload the software from the computer onto at least one of the external programmers over the wireless radio frequency link in response to determining that the at least one of the external programmers is without the software thereby eliminating the need for manual insertion of a portable media into each external programmer in order to transfer the software.

27. The computer program product of claim 26, wherein the computer is portable and comprises at least one of the following:
a handheld computing device;
a personal digital assistant (PDA);
a laptop computer; and
a pocket personal computer.

28. The computer program product of claim 26, wherein the computer readable program code for causing the computer to upload the software from the transfer computing device includes computer readable program code for causing the computer to transmit the software to the external programmers in succession without manual intervention and without interrupting any functionality of the external programmers interfacing with an implantable medical device.

29. The computer program product of claim 26, wherein the computer readable code for causing the computer to determine whether any of the external programmers are without the software includes computer readable code for causing the computer to:
transmit an inquiry to each of the external programmers requesting whether any version of the software is presently stored on the external programmer inquired;
receive a response from each external programmer inquired as to what version is presently stored on the external programmer; and
determine from each response, which of the external programmers is presently one of not storing any version of the software and storing a version of the software different from the software to be uploaded.

30. A computer program product comprising a computer usable medium having control logic stored therein for causing external programmers for implantable medical devices to receive software via a wireless RF link from a computing device, the control logic comprising computer readable program code for causing the external programmers to:
receive communication from the computing device via the wireless RF link;
prepare to receive an upload of the software via the wireless RF link;
receive the upload of the software from the computing device via the wireless RF link; and
store the software onto a memory of each external programmer receiving the upload of software.

31. The computer program product of claim 30, further comprising computer readable program code for causing the external programmers to:
replace the version of the software currently stored with the upload of the software; and
utilize the software while in communication with the implantable medical devices.

32. The computer program product of claim 30, wherein the computer readable program code for causing the external programmers to prepare to receive the upload of software comprises computer readable program code for causing the external programmers to:
in response to receiving the communication, detect at least one of whether any version of the software and what version of the software is currently stored on each external programmer receiving the communication; and
communicate to the computing device via the wireless RF link at least one of whether any version of the software and what version of the software is currently stored on each external programmer receiving the communication.

33. The computer program product of claim 30, further comprising computer readable program code for causing the external programmers to:
at least one of monitor the wireless RF communications link and receive notice of a wireless RF communication; and
in response to receiving the notice, establish communication with the computing device over the wireless RF link.

34. A system for transferring software from a computing device onto one or more external programmers for implantable medical devices over a wireless radio frequency (RF) link, the system comprising:
at least one external programmer operative to:
receive communication from the computing device via the wireless RF link;
in response to receiving the communication, detect at least one of whether any version of the software and what version of the software is currently stored on the at least one external programmer receiving the communication; and
communicate to the computing device via the wireless RF link at least one of whether any version of the software and what version of the software is currently stored on the at least one external programmer receiving the communication;
receive the upload of the software from the computing device via the wireless RF link; and
store the software onto a memory of each external programmer receiving the upload of software; and
at least one computing device operative to:
establish communication with the at least one external programmer via the wireless RF link;
determine whether the at least one external programmer is without the software, wherein the software comprises at least one of external programmer instructions and data; and
upload the software onto the at least one external programmer via the wireless radio frequency link in response to determining that the at least one external programmer is without the software thereby eliminating the need for manual insertion of a portable media into each external programmer in order to transfer the software.

35. The system of claim 34, wherein the computing device is portable and comprises at least one of the following:
a handheld computing device;
a personal digital assistant (PDA);
a laptop computer; and
a pocket personal computer further comprising at least one of a server computer and a second portable computing device wherein the computing device is operative to:
access at least one of a wired network and a second wireless RF link to retrieve the software from at least one of the server computer and the second portable computing device; and
download the software onto the computing device from at least one of the server computer and the second portable computing device via at least one of the wired network and the second wireless RF link.
